(19) United States
(12) Patent Application Publication
(10) Pub. No.: US 2026/0115212 A1
(43) Pub. Date: Apr. 30, 2026

(54) PROCESS FOR THE PURIFICATION OF CANNABIDIOL FROM HERBAL MATERIAL

(71) Applicant: A2W Pharma Limited

(72) Inventors: Domenico GURRADO, Marsaxlokk (MT); Fayuan HU, Marsaskala (MT)

(21) Appl. No.: 18/927,554

(22) Filed: Oct. 25, 2024

(51) Int. Cl. A61K 31/00; A61K 36/185; C07C 65/19; C07D 211/62

(52) U.S. Cl. CPC A61K 31/658; A61K 36/3482; C07C 65/19; C07D 211/62; A61K 2236/33; A61K 2236/35; C07B 2200/13

(57) ABSTRACT

The present invention relates to a process for purifying cannabinoids, specifically cannabidiol, in a substantially pure form from herbal material. This process involves a sequence of purification steps including winterization, co-crystallization, dissociation, decarboxylation, and crystallization. Cannabidiol is purified from an extract of herbal material containing approximately 50-70% cannabidiolic acid (CBDA) and 10-20% cannabidiol (CBD), along with other cannabinoids. The substantially pure form of cannabidiol can be used in medicaments for treating various diseases or conditions. The invention also relates to co-crystals of L-proline and cannabidiolic acid and their use in obtaining substantially pure cannabidiol free from tetrahydrocannabinol (THC).

### FIELD

The invention relates to methods for purifying cannabidiol in a substantially pure form from herbal material. Specifically, the invention pertains to co-crystals of cannabidiolic acid with L-proline, processes for their production, and their use in the purification of cannabidiol.

### BACKGROUND

Cannabis has long been recognized for its therapeutic properties, used by ancient civilizations like the Egyptians, Greeks, and Chinese. Cannabis refers to a group of plants (Cannabis sativa, Cannabis indica, and Cannabis ruderalis) known for their psychoactive and medicinal effects. The plant contains over 120 cannabinoids, including cannabidiol (CBD) and tetrahydrocannabinol (THC), which interact with the human endocannabinoid system, influencing the immune and central nervous systems.

Among cannabinoids, CBD is particularly valued for its anti-inflammatory and anxiolytic effects, without the psychoactive properties of THC. The main cannabinoid acids in cannabis are Δ9-tetrahydrocannabinolic acid (Δ9-THCA) and cannabidiolic acid (CBDA), which can be converted into Δ9-THC and CBD, respectively. Given CBD's potential, there is a demand for substantially pure CBD, free from psychoactive contaminants like THC.

Traditional methods of purifying cannabinoids, such as solvent extraction and chromatography, are often costly and complex. While various techniques have been developed to isolate CBD, many processes remain inefficient, yielding products with significant levels of THC and other impurities. There is a clear need for a simpler, more cost-effective method to obtain high-purity CBD, particularly for pharmaceutical applications where the presence of THC is undesirable.

The present invention addresses this need by offering a novel process to purify CBD from cannabis, resulting in a substantially pure product that is suitable for use in medical formulations. This method is designed to be scalable, cost-effective, and highly selective for CBD, providing a valuable alternative to existing purification techniques.

### BRIEF DESCRIPTION OF DRAWINGS

FIG. 1 shows ¹³C CPMAS SSNMR spectra comparison between CBDA-Proline, L-proline, and ¹³C NMR of pure CBDA

FIG. 2 shows a typical chromatogram of an extract of Example 1.

FIG. 3 shows a typical chromatogram of winterized extract of Example 2.

FIG. 4 shows a typical chromatogram of a co-crystal of Example 3.

FIG. 5 shows a typical chromatogram after dissociation of Example 4.

FIG. 6 shows a typical chromatogram after crystallization of Example 6.

### SUMMARY OF INVENTION

The present invention relates to a process for purifying cannabinoids, particularly cannabidiol, in a substantially pure form from herbal material. The process involves a sequence of steps including winterization, co-crystallization, dissociation, decarboxylation, and crystallization.

A method of purifying cannabidiol comprises:
Extracting the herbal material with a solvent or supercritical carbon dioxide to obtain an extract containing cannabidiolic acid (CBDA).
Subjecting the extract to winterization using at least one solvent to remove non-polar constituents, followed by distillation to remove residual organic solvent.
Co-crystallizing CBDA with L-proline from an aliphatic solvent to obtain crystalline CBDA-L-proline co-crystals.
Dissociating CBDA from the co-crystals using—aliphatic solvents and water.
Decarboxylating CBDA into cannabidiol by heating the dissociated material.
Purifying the dissociated material through crystallization to obtain substantially pure crystalline cannabidiol.

The substantially pure form of cannabidiol can be used in medicaments for treating various diseases or disorders, such as, Lennox Gastaut Syndrome, Dravet Syndrome, myoclonic seizures, schizophrenia, juvenile spasms, refractory infantile spasms, tubular sclerosis complex, brain tumours, neuropathic pain, cannabis use disorder, post-traumatic stress disorder, anxiety, early psychosis, Alzheimer's Disease autism. The invention also relates to the use of the co-crystals of L-proline and cannabidiolic acid in obtaining substantially pure cannabidiol free from tetrahydrocannabinol (THC).

### DETAILED DESCRIPTION OF THE INVENTION

The invention relates to a process for purifying cannabinoids, particularly cannabidiol, in a substantially pure form from herbal material. The process involves a sequence of steps including winterization, co-crystallization, dissociation, decarboxylation, and crystallization. A method of purifying cannabidiol comprises:
Extracting the herbal material with a solvent or supercritical carbon dioxide to obtain an extract containing cannabidiolic acid (CBDA).
Subjecting the dissociated material to winterization using at least one solvent to remove non-polar constituents, followed by distillation to remove residual organic solvent.
Co-crystallizing CBDA with L-proline from an aliphatic solvent to obtain crystalline CBDA-L-proline co-crystals.
Dissociating CBDA from the co-crystals using aliphatic solvents and water
Decarboxylating CBDA into cannabidiol by heating the dissociated material.
Purifying the dissociated material through crystallization to obtain substantially pure crystalline cannabidiol.

The process according to the present invention allows for the selective purification of cannabidiol from herbal material. The purification method is less expensive and simpler compared to existing chromatographic techniques, making it suitable for large-scale production. The invention also relates to the use of co-crystals of L-proline and cannabidiolic acid in obtaining substantially pure cannabidiol free from tetrahydrocannabinol (THC).

All terms used herein, unless otherwise stated, shall be understood in their ordinary meaning as known in the art. Specific definitions for certain terms used in the present application are set forth below and are intended to apply uniformly throughout the specification and claims unless a broader definition is expressly set out.

For the purposes of the present invention, any ranges provided include both the lower and upper endpoints. Ranges and values given, such as temperatures and times, should be considered approximate unless specifically stated otherwise.

The term “substantially pure cannabidiol” as used herein refers to cannabidiol having a purity of at least 98.0% by weight, preferably at least 99.0% by weight, as determined by HPLC analysis. The term “herbal material” as used herein refers to cannabis plant material, including any part of the plant Cannabis sativa L., preferably flowers, leaves, stems, or a mixture thereof.

For the purposes of the invention, the term “cocrystal” refers to a crystalline entity with at least two different components (also known as “conformers”) constituting the unit cell at room temperature (20-25° C.) and interacting through weak interactions. In a cocrystal, one component crystallizes with one or more neutral components. The cocrystals may include one or more solvent molecules in the crystal lattice.

The extraction step can be carried out using various organic solvents, such as ethanol, methanol, isopropanol, hexane, heptane, or a mixture thereof. The extraction step can be carried out also using the supercritical fluid extraction technique (CO2).

The winterization step is carried out using alcohols, such as ethanol, methanol, or a mixture thereof, at a temperature of about −20° C. to about 5° C., preferably at about 0° C.

The co-crystallization step is carried out using an aliphatic solvent, such as heptane, n-heptane, n-hexane, or a mixture thereof. The preferred solvent is n-heptane.

The decarboxylation step is carried out by heating the dissociated material at a temperature of about 80° C. to about 150° C., preferably at about 130° C.

The crystallization step is carried out using a suitable solvent, such as n-heptane, n-hexane, or a mixture thereof. The preferred solvent is n-heptane.

The substantially pure cannabidiol obtained by the process according to the present invention can be used in various pharmaceutical formulations, such as oral, topical, or inhalable dosage forms. The cannabidiol can be formulated into solutions, tablets, capsules, creams, ointments, gels, or inhalable aerosols.

The invention also relates to a method of treating various diseases or conditions in a subject in need thereof, comprising administering to the subject an effective amount of substantially pure cannabidiol obtained by the process according to the present invention. The diseases or conditions include, but are not limited to, epilepsy, anxiety, pain, inflammation, and neurodegenerative disorders such as, Lennox Gastaut Syndrome, Dravet Syndrome, myoclonic seizures, schizophrenia, juvenile spasms, refractory infantile spasms, tubular sclerosis complex, brain tumours, neuropathic pain, cannabis use disorder, post-traumatic stress disorder, anxiety, early psychosis, Alzheimer's Disease autism.

In one embodiment of the invention, the extraction of cannabinoids from the extracted herbal material is performed using supercritical CO2. The process is carried out at a temperature ranging from about 50° C. to about 100° C., preferably from about 50° C. to about 60° C. The pressure is maintained between about 250 bar and about 280 bar, with a preferred pressure of about 265 bar. The extraction process allows for the separation of cannabinoids, particularly cannabidiolic acid (CBDA), from other plant constituents. The use of supercritical CO2 is advantageous because it is non-toxic, environmentally friendly, and can be easily removed from the final product.

Following extraction, the winterization step involves the removal of non-polar constituents such as fats, waxes, and chlorophylls. This is achieved by dissolving the dissociated material in alcohols a, such as ethanol, methanol, or a mixture thereof. The preferred solvent is ethanol. The solution is then cooled to a temperature between about −20° C. and 5° C., preferably around 0° C. The cooling causes the non-polar constituents to precipitate out of the solution, which can then be removed by filtration or centrifugation. The winterized solution is then subjected to distillation to remove any residual organic solvents, leaving behind a concentrated extract rich in CBDA.

The next step in the process is the co-crystallization of CBDA with L-proline. This is carried out by dissolving CBDA in an aliphatic solvent, such as heptane, n-heptane, n-hexane or a mixture thereof, with n-heptane being the preferred solvent. L-proline is then added to the solution, resulting in the formation of CBDA-L-proline co-crystals. The co-crystallization process can be carried out at room temperature, and the resulting co-crystals are isolated by filtration. The co-crystallization step is crucial as it allows for the selective crystallization of CBDA, while leaving behind impurities such as tetrahydrocannabinol (THC).

Once the CBDA-L-proline co-crystals are obtained, the next step is the dissociation of CBDA from the co-crystals. This is achieved dissolving at 50-60° C., preferably at 55° C. the co-crystal in an aliphatic solvent such as heptane, n-heptane, n-hexane or a mixture thereof, with n-heptane being the preferred solvent. After the dissolution, the dissociated L-Proline is extracted with water. The liquid phases are separated and the organic phase, rich in CBDA is heated at 40-50° C., preferably at 45° C. under vacuum, to remove the solvent leaving behind a concentrated purified extract rich in CBDA.

The decarboxylation step involves the conversion of CBDA into cannabidiol (CBD). This is accomplished by heating the CBDA-rich purified extract to a temperature between about 80° C. and 150° C., preferably around 130° C., for a period sufficient to complete the decarboxylation reaction. The heating causes the carboxyl group to be removed from CBDA, resulting in the formation of CBD. The decarboxylation process is monitored to ensure complete conversion of CBDA to CBD, which can be confirmed using HPLC or other suitable analytical techniques.

The final step in the purification process is the crystallization of cannabidiol to obtain it in a substantially pure form. The decarboxylated material, which now contains CBD, is dissolved in a suitable solvent such as heptane, hexane, or a mixture thereof, with n-heptane being the preferred solvent. The solution is then cooled to induce crystallization of CBD. The crystalline CBD is isolated by filtration, washed with cold solvent to remove any residual impurities, and dried to yield substantially pure CBD with a purity of at least 98.0% by weight, preferably at least 99.0% by weight, as determined by HPLC analysis.

One aspect of the present invention provides a process for preparing an herbal extract of cannabidiol in both forms: CBDA acid (approximately 50-70%) and CBD neutral (approximately 10-20%). This herbal extract can be obtained through supercritical carbon dioxide extraction, with or without an organic solvent modifier, or by extraction with an organic solvent from the herbal material.

In another aspect, the present invention provides an herbal extract of cannabidiol in both forms (CBDA acid, around 45-65%, and CBD neutral, around 10-20%) isolated according to the process outlined above.

The below table provides the HPLC assay % w/w of a typical chromatogram of the herbal extract.

| | |
|---|---|
| Cannabidiol (CBD) % w/w | 12.40 |
| Cannabidiolic Acid (CBDA) % w/w | 61.11 |
| Δ9-Tetrahydrocannadinol (THC) % w/w | ND |
| Δ9-Tetrahydrocannadinolic Acid (THCA) % | 0.86 |
| w/w | |
| Cannabidivarin (CBDV) area % | 0.73 |
| Cannabidibutol (CBD-C4) area % | 0.27 |
| Cannabidiol Hydroxy Quinone (CBDQ) area % | ND |
| Cannabinol (CBN) area % | 0.13 |
| Cannabigerol (CBG) area % | 0.38 |

In a further aspect, the present invention provides a process for obtaining a substantially pure form of cannabidiol (CBD) from the herbal extract, which method comprises:
(a) obtaining an extract of herbal material containing approximately 50-70% CBDA acid and 10-20% CBD neutral,
(b) winterizing the extract of herbal material to remove waxes,
(c) co-crystallizing the winterized extract with L-proline to form co-crystals,
(d) dissociating the co-crystals to obtain an oil,
(e) decarboxylating the oil to obtain decarboxylated oil, and
(f) crystallizing the decarboxylated oil to obtain substantially pure cannabidiol

In an aspect, the present invention provides a substantially pure form of cannabidiol with HPLC purity of not less than 99.0% and assay between 98.0 and 102% w/w.

In a further aspect, the present invention provides a substantially pure form of cannabidiol wherein two impurities, namely cannabidiolic acid (CBDA) and delta-9 tetrahydrocannabinol (Δ9THC), are below the limit of detection i.e., <0.01% by HPLC-UV.

In an additional aspect, the present invention provides a substantially pure form of cannabidiol which is free from tetrahydrocannabinol which is not more than (NMT) limit of detection (LOD being 0.01%)

In another aspect, the present invention provides a substantially pure form of cannabidiol comprising ≤0.5% of the impurities cannabidivarin (CBDV) and cannabidiol-C4 (CBD-C4), respectively by HPLC.

In one of the aspects the present invention refers to cocrystals of CBDA and a conformer i.e. 2,4-dihydroxy-3-[(1R,6R)-3-methyl-6-prop-1-en-2-ylcyclohex-2-en-1-yl]-6-pentylbenzoic acid (cannabidiolic acid or CBDA) and a zwitterionic conformer.

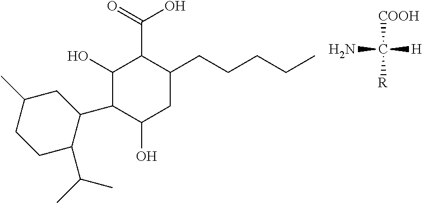

wherein R is a normal or branched alkyl group having 1 to 10 carbon atoms. And independently are hydrogen or a substituted or unsubstituted normal or branched alkyl group having 1 to 6 carbon atoms and the unsubstituted normal or branched alkyl group may include a nitrogen atom which is a part of a heterocyclic group having three to seven carbon atoms, the individual carbon atoms of the heterocyclic group independently being substituted or unsubstituted.

The present invention provides a co-crystal of 2,4-dihydroxy-3-[(1R,6R)-3-methyl-6-prop-1-en-2-ylcyclohex-2-en-1-yl]-6-pentylbenzoic acid (cannabidiolic acid or CBDA) wherein the R group of the zwitterionic conformer is L-proline.

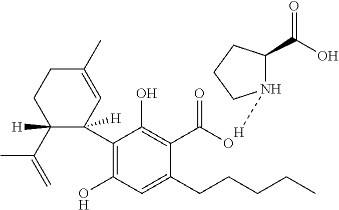

The present invention provides a co-crystal of 2,4-dihydroxy-3-[(1R,6R)-3-methyl-6-prop-1-en-2-ylcyclohex-2-en-1-yl]-6-pentylbenzoic acid (cannabidiolic acid or CBDA) and L-proline characterized by Solid-State NMR as depicted in FIG. 1.

In one of the aspects the present invention provides the use of a co-crystal of 2,4-dihydroxy-3-[(1R,6R)-3-methyl-6-prop-1-en-2-ylcyclohex-2-en-1-yl]-6-pentylbenzoic acid (cannabidiolic acid or CBDA) and L-proline in the purification of cannabinoids to obtain a substantially pure form of cannabidiol.

The present invention is further described by the examples given below. These examples are provided only to illustrate the invention and should not be construed to limit the scope of the invention.

The starting herbal extract of the present invention can be obtained through supercritical carbon dioxide extraction or extraction with solvents like ethanol. The herbal extract of the present invention contains cannabidiol in both forms: CBDA acid (around 50-70%) and CBD neutral (around 10-20%).

### EXAMPLES

### Example 1: Obtaining an Extract of Herbal Material

Composition: The extract contains approximately 45-65% cannabidiolic acid (CBDA) and 10-20% cannabidiol (CBD).

| | |
|---|---|
| CBD % w/w | 12.40 |
| CBDA % w/w | 61.11 |
| THC % w/w | ND |
| THCA % w/w | 0.86 |
| CBDV area % | 0.73 |
| CBD-C4 | 0.27 |
| area % | |
| CBDQ area % | ND |
| CBN area % | 0.13 |
| CBG area % | 0.38 |

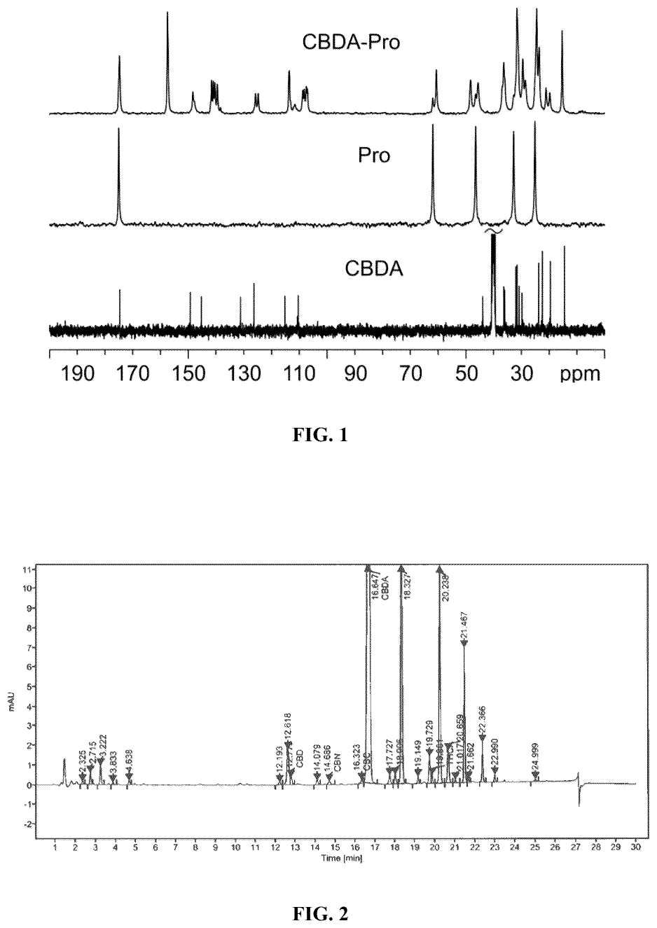

The initial herbal extract primarily contains CBDA (61.11% w/w) and a smaller amount of CBD (12.40% w/w), with minimal THC (not detectable, ND) and lower amounts of other cannabinoids. This high CBDA content in the extract forms the basis for the subsequent purification steps, ensuring that the process starts with a significant quantity of the desired cannabinoid. FIG. 2 shows a typical chromatogram of an extract.

### Example 2: Winterization of Herbal Extract

1. Dissolution: Add 63 kg of ethanol to 50 kg of herbal extract (equivalent to 33 kg of CBD) and heat the mixture at 40° C. until complete dissolution occurs.
2. Cooling and Filtration: Cool the mixture to 0-5° C., maintain it at this temperature for 1 hour, and then filter.
3. Yield: Wash the cake with cold ethanol and evaporate the filtrate to yield 48 kg of winterized oil (CBD eq. 65%, i.e., 31 kg CBD).

After winterization, non-polar constituents like fats and waxes are removed, resulting in a concentrated extract with enriched CBDA and reduced impurities like THCA (0.86% w/w).

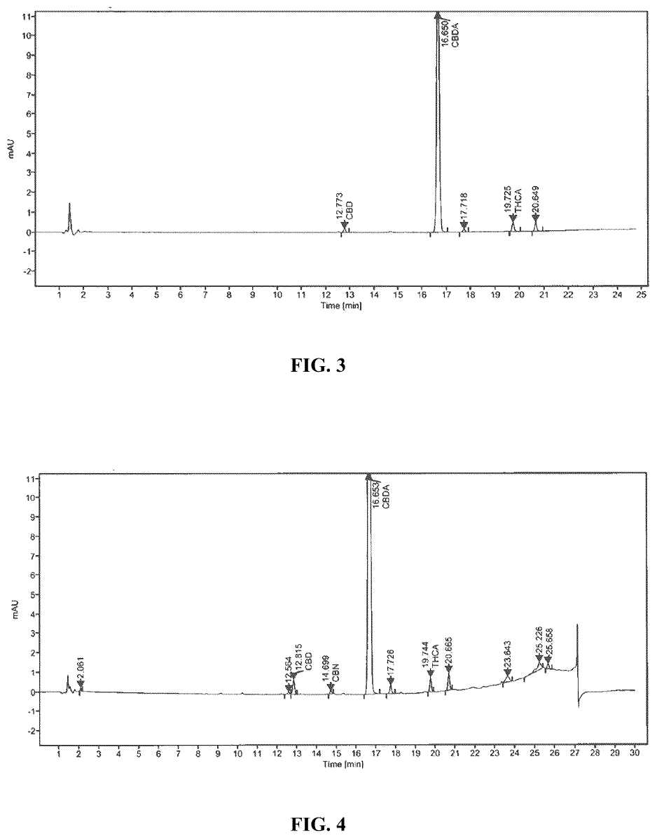

The reduction in impurities and the concentration of CBDA are essential for effective co-crystallization, ensuring the process selectively isolates the desired cannabinoid. FIG. 3 shows a typical chromatogram of winterized oil

### Example 3: First Co-Crystallization with L-Proline

1. Mixing: Add 200 kg of n-heptane to 48 kg of winterized oil from Step (a) and then add 12 kg of L-proline.
2. Stirring: Stir the mixture at room temperature (20-25° C.) for 12-20 hours.
3. Filtration: Filter the slurry and wash the cake with n-heptane.
4. Drying: Dry the wet solid in an oven at 40° C. under vacuum to yield 40-50 kg of co-crystallized material (CBD eq. 65%, i.e., 30 kg CBD). Concentrate the filtrate (mother liquor) at 45° C. under vacuum to obtain the second crop (CBD eq. 54%, i.e., 11 kg CBD).

The co-crystallization step selectively isolates CBDA, which is evident in the formation of CBDA-L-proline co-crystals, reducing the presence of other cannabinoids and impurities like THC.

This step is crucial for eliminating THC and other undesirable components, resulting in a purified form of CBDA ready for further processing. FIG. 4 shows a typical chromatogram of a co-crystal.

### Example 4: Dissociation of Co-Crystals

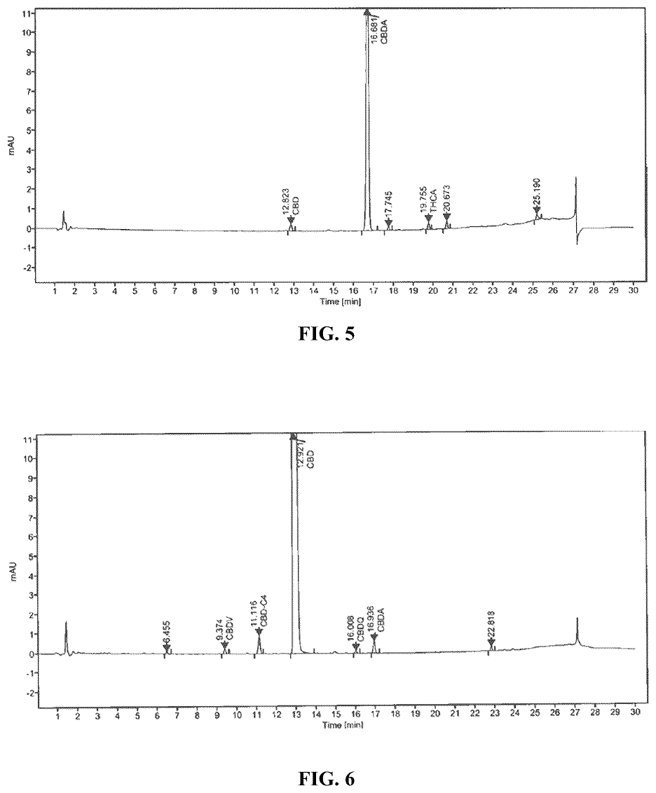

1. Mixing: Add 400 kg of n-heptane to 50 kg of co-crystals from Step (c) and then add 200 kg of water.
2. Stirring: Stir the mixture at 50-55° C. After 1 hour, the co-crystals should be completely dissolved.
3. Phase Separation: Cool the reaction mass to room temperature and separate the phases. Discard the aqueous phase and wash the organic phase with two aliquots of 250 L water each.
4. Concentration: Concentrate the organic phase at 45° C. under vacuum to yield 60-70 kg of dissociated oil (30-32 Kg of CBD). FIG. 5 shows a typical chromatogram of dissociated oil

### Example 5: Decarboxylation

1. Heating: Transfer 60-70 kg of dissociated oil from Step (d) to a reactor and heat at 130-135° C. for 4-5 hours under a nitrogen atmosphere.
2. Cooling: Cool the reaction mass to room temperature to yield 40-50 kg of decarboxylated oil (CBD eq. 70-75%, i.e., 30 kg CBD).

After decarboxylation, the chromatogram shows a significant increase in CBD content as CBDA is converted to CBD. The CBD percentage increases, and other cannabinoid impurities remain low or undetectable.

The successful conversion of CBDA to CBD and the maintenance of low impurity levels highlight the effectiveness of the decarboxylation process in producing high-purity cannabidiol.

### Example 6: Crystallization

1. Dissolution: Add 22 kg of n-heptane to 30 kg of decarboxylated oil. Heat the mixture to 45-50° C. and stir for 15 minutes until dissolution occurs.
2. Crystallization: Cool the reaction mass to 0-5° C. and seed with 5-10 g of pure CBD (provide the source of this material). Stir for 2 hours.
3. Filtration and Drying: Filter the slurry and dry the wet solid at 40° C. under vacuum to yield 18-20 kg of crystallized material (CBD eq. 99%).
4. Recrystallization: Add the 14 kg of crystallized material to 17 kg of n-heptane, heat to 45-50° C., and stir for 15 minutes until dissolution occurs. Cool to 0-5° C., seed with 3-5 g of pure CBD, and stir for 2 hours. Filter to obtain 15-18 kg of pure CBD.

The final crystallized product shows substantially pure CBD with a purity of at least 99.0% w/w, as determined by HPLC, with non-detectable levels of THC and minimal amounts of other cannabinoids (e.g., CBDV at 0.1% area, CBD-C4 at 0.3% area). FIG. 6 shows a typical chromatogram of pure CBD after crystallization.

The final crystallization ensures that the cannabidiol obtained is of the highest purity, free from significant impurities, and suitable for pharmaceutical applications.

This process achieves a substantially pure form of cannabidiol with an HPLC purity of not less than 99.0%, ensuring the final product is nearly free of THC and other undesirable cannabinoids, making it ideal for therapeutic use. The co-crystallization with L-proline, followed by careful dissociation and crystallization, offers a unique and cost-effective approach to cannabinoid purification that is more scalable than traditional methods.

The substantially pure cannabidiol obtained through this process can be used in various pharmaceutical formulations, including oral dosage forms such as tablets and capsules, topical forms like creams and ointments, and inhalable aerosols. These formulations are suitable for treating a wide range of conditions, and disorders such as, Lennox Gastaut Syndrome, Dravet Syndrome, myoclonic seizures, schizophrenia, juvenile spasms, refractory infantile spasms, tubular sclerosis complex, brain tumours, neuropathic pain, cannabis use disorder, post-traumatic stress disorder, anxiety, early psychosis, Alzheimer's Disease autism. The high purity of cannabidiol ensures that the pharmaceutical products are free from undesirable impurities like THC, enhancing their safety and effectiveness for therapeutic use.

The purification process described in this invention offers several advantages over conventional methods. Firstly, the use of liquid CO2 for dissociation is environmentally friendly, non-toxic, and allows for the selective separation of cannabinoids. Secondly, the co-crystallization of CBDA with L-proline is a novel technique that enhances the purity of the final CBD product by selectively isolating CBDA and excluding impurities like THC. Thirdly, the overall process is simpler and more cost-effective than chromatographic techniques, making it suitable for large-scale production. Ultimately, this process yields substantially pure cannabidiol, ideal for use in pharmaceutical formulations for treating multiple medical conditions.

What is claimed is:

1. A process for purifying cannabidiol from herbal material, the process comprising:
extracting the herbal material with a solvent or supercritical carbon dioxide to obtain an extract containing approximately 50-70% cannabidiolic acid (CBDA);
winterizing the extract using at least one solvent at a temperature ranging from about −20° C. to about 5° C. to remove non-polar constituents, followed by distillation to remove residual organic solvent;
co-crystallizing CBDA with L-proline from an aliphatic solvent to obtain crystalline CBDA-L-proline co-crystals;
dissociating CBDA from the co-crystals using an aliphatic solvent and water at a temperature ranging from about 50° C. to about 60° C. and removing the conformer and distilling the solvent;
decarboxylating CBDA into cannabidiol by heating the dissociated material to a temperature ranging from about 80° C. to about 150° C.;
purifying the dissociated material through crystallization using a suitable solvent to obtain substantially pure crystalline cannabidiol with a purity of at least 99.0% by weight and 99.0% by area %.

2. The process according to claim 1, wherein the solvent used for extracting the herbal material is selected from the group consisting of ethanol, methanol, isopropanol, hexane, heptane, and mixtures thereof.

3. The process according to claim 1, wherein the herbal material is extracted using supercritical carbon dioxide at a pressure ranging from 250-280 bar and a temperature ranging from 50° C. to 60° C.

4. The process according to claim 1, wherein the solvent used in the winterization step is selected from the group consisting of alcohols such as ethanol, methanol, and mixtures thereof.

5. The process according to claim 1, wherein the aliphatic solvent used for co-crystallization is selected from the group consisting of hexane, heptane, and mixtures thereof.

6. The process according to claim 1, wherein the substantially pure cannabidiol obtained has a purity of at least 99.0% by weight and 99.0% by area % as determined by HPLC analysis.

7. The process according to claim 1, wherein the substantially pure cannabidiol obtained is free from tetrahydrocannabinol (THC) and other impurities, with THC content below the limit of detection (<0.01% by HPLC-UV).

8. A pharmaceutical composition comprising substantially pure cannabidiol obtained by the process according to claim 1, and a pharmaceutically acceptable carrier and/or excipient.

9. The pharmaceutical composition according to claim 8, wherein the composition is formulated for oral, topical, or inhalable administration.

10. The pharmaceutical composition according to claim 9, wherein the composition is in the form of a solution, tablet, capsule, cream, ointment, gel, or inhalable aerosol.

11. A method of treating a disease or condition in a subject in need thereof, comprising administering to the subject an effective amount of substantially pure cannabidiol obtained by the process according to claim 1.

12. The method according to claim 11, wherein the disease or condition is selected from the group consisting of epilepsy, anxiety, pain, inflammation, and neurodegenerative disorders such as Lennox Gastaut Syndrome, Dravet Syndrome, myoclonic seizures, schizophrenia, juvenile spasms, refractory infantile spasms, tubular sclerosis complex, brain tumors, neuropathic pain, cannabis use disorder, post-traumatic stress disorder, anxiety, early psychosis, Alzheimer's Disease, and autism.

13. A co-crystal of 2,4-dihydroxy-3-[(1R,6R)-3-methyl-6-prop-1-en-2-ylcyclohex-2-en-1-yl]-6-pentylbenzoic acid (cannabidiolic acid or CBDA) and L-proline.

14. The co-crystal according to claim 13, characterized by Solid-State NMR as depicted in FIG. 1.

15. A method of purifying cannabinoids to obtain a substantially pure form of cannabidiol, comprising using a co-crystal of 2,4-dihydroxy-3-[(1R,6R)-3-methyl-6-prop-1-en-2-ylcyclohex-2-en-1-yl]-6-pentylbenzoic acid (cannabidiolic acid or CBDA) and L-proline in the purification process.
